(19) United States
(12) Patent Application Publication
(10) Pub. No.: US 2016/0022609 A1
(43) Pub. Date: Jan. 28, 2016

(54) METHODS FOR TREATING CHRONIC PAIN USING A COMBINATION OF BUPROPION ((±)-2-(TERT-BUTYLAMINO)-1-(3-CHLOROPHENYL)PROPAN-1-ONE) AND PHENTERMINE (2-METHYL-1-PHENYLPROPAN-2-AMINE)

(71) Applicant: Thomas R. Winston, Leawood, KS (US)

(72) Inventor: Thomas R. Winston, Leawood, KS (US)

(21) Appl. No.: 14/337,653

(22) Filed: Jul. 22, 2014

(51) Int. Cl. A61K 31/137

(52) U.S. Cl. CPC A61K 31/137

(57) ABSTRACT

Methods for treating chronic pain are disclosed. More specifically, methods for treating chronic pain include administering a composition including bupropion and phentermine Methods for treating neuropathic pain and for treating nociceptive pain are further disclosed.

### BACKGROUND OF THE DISCLOSURE

The present disclosure is generally directed to methods for treating chronic pain. Methods for treating chronic pain include administering to an individual in need a composition including a combination of bupropion and phentermine.

Chronic pain is characterized by pain that lasts longer than three to six months. Chronic pain can also be characterized as pain that extends beyond the expected period of healing. Chronic pain can be divided into nociceptive pain and neuropathic pain. Nociceptive chronic pain is caused by activation of nociceptors. Neuropathic pain is caused by damage to or the malfunction of the nervous system. Neuropathic pain can be further divided into peripheral neuropathic pain and central neuropathic pain.

Chronic pain has been characterized as a disease affecting brain structure and function. Persistent pain can lead to grey matter loss and brain activity is altered in individuals suffering from chronic pain. Methods for treating chronic pain can reduce suffering and improve quality of life. Accordingly, there exists a need for treating chronic pain.

### SUMMARY OF THE DISCLOSURE

The present disclosure is generally directed to methods for treating chronic pain. More specifically, in one aspect, the present disclosure is directed to a method for treating chronic pain in an individual in need comprising administering to the individual a composition including a combination of bupropion and phentermine

In another aspect, the present disclosure is directed to a method for treating neuropathic pain in an individual in need comprising administering to the individual a composition including a combination of bupropion and phentermine.

In another aspect, the present disclosure is directed to a method for treating nociceptive pain in an individual in need comprising administering to an individual in need a composition including a combination of bupropion and phentermine

### DETAILED DESCRIPTION

While the disclosure is susceptible to various modifications and alternative forms, specific embodiments thereof are herein described below in detail. It should be understood, however, that the description of specific embodiments is not intended to limit the disclosure to cover all modifications, equivalents and alternatives falling within the spirit and scope of the disclosure as defined by the appended claims.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, suitable methods and materials are described below.

Methods for Treating Pain

In one aspect, the present disclosure is directed to a method for treating chronic pain in an individual in need. The method includes administering to an individual in need a composition including a combination of bupropion and phentermine

In another embodiment, the method for treating chronic pain in an individual in need can further include administering methylphenidate (methyl phenyl(piperidin-2-yl)acetate, CONCERTA® and RITALIN®), which can increase norepinephrine, epinephrine and dopamine in the brain.

In yet another embodiment, the method for treating chronic pain in an individual in need can further include administering atomoxetine ((3R)-N-methyl-3-(2-methylphenoxy)-3-phenylpropan-1-amine; (R)-N-methyl-3-phenyl-3-(o-tolyloxy)propan-1-amine; STRATTERA®). Atomoxetine is approved by the FDA for the treatment of ADHD and selectively inhibits norepinephrine reuptake. Suitable doses of atomxetine can be from about 5 mg to about 100 mg.

In another aspect, the present disclosure is directed to a method for treating neuropathic pain in an individual in need. The method includes administering to an individual in need a composition including a combination of bupropion and phentermine.

In another embodiment, the method for treating neuropathic pain in an individual in need can further include administering methylphenidate (methyl phenyl(piperidin-2-yl)acetate, CONCERTA® and RITALIN®), which can increase norepinephrine, epinephrine and dopamine in the brain.

In yet another embodiment, the method for treating neuropathic pain in an individual in need can further include administering atomoxetine ((3R)-N-methyl-3-(2-methylphenoxy)-3-phenylpropan-1-amine; (R)-N-methyl-3-phenyl-3-(o-tolyloxy)propan-1-amine; STRATTERA®). Atomoxetine is approved by the FDA for the treatment of ADHD and selectively inhibits norepinephrine reuptake. Suitable doses of atomxetine can be from about 5 mg to about 100 mg.

In another aspect, the present disclosure is directed to a method for treating nociceptive pain in an individual in need. The method includes administering to an individual in need a composition including a combination of bupropion and phentermine.

In another embodiment, the method for treating nociceptive pain in an individual in need can further include administering methylphenidate (methyl phenyl(piperidin-2-yl)acetate, CONCERTA® and RITALIN®), which can increase norepinephrine, epinephrine and dopamine in the brain.

In yet another embodiment, the method for treating nociceptive pain in an individual in need can further include administering atomoxetine ((3R)-N-methyl-3-(2-methylphenoxy)-3-phenylpropan-1-amine; (R)-N-methyl-3-phenyl-3-(o-tolyloxy)propan-1-amine; STRATTERA®). Atomoxetine is approved by the FDA for the treatment of ADHD and selectively inhibits norepinephrine reuptake.

Without being bound by theory, bupropion (formula I; (±)-2-(tert-butylamino)-1-(3-chlorophenyl)propan-1-one) is known as an antidepressant that functions as a mild dopamine reuptake inhibitor and weak norepinephrine reuptake inhibitor. Bupropion also functions as a nicotinic acetylcholine receptor antagonist.

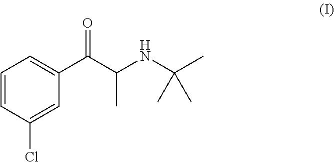 [SMILES: CC(NC(C)(C)C)C(=O)c1cccc(Cl)c1]

Without being bound by theory, phentermine acts on the hypothalamus portion of the brain to stimulate the adrenal glands to release norepinephrine. Phentermine can also stimulate the release of epinephrine or adrenaline.

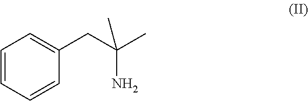

Bupropion as a single agent is known for treating Attention Deficit Hyperactivity Disorder (ADHD) at a dose of 400 mg per day. Phentermine is known as an appetite-suppressant for which the U.S. Food and Drug Administration recommends a dosage of a half of a single 37.5 mg phentermine hydrochloride tablet up to a single 37.5 mg phentermine hydrochloride tablet (equivalent to 30 mg phentermine base) for patients with a body mass index of greater than 30 kg/m² (or greater than 27 kg/m² with other risk factors such as controlled hypertension, diabetes, and hyperlipidemia). Because of the development of tolerance, administration of phentermine for appetite-suppression is recommended for up to 12 weeks.

Administering the composition of the present disclosure allows for lower initial dosages of bupropion and/or phentermine than their dosages when administered as single agents. The lower initial dosages can contribute to a reduction in acquired tolerance. The lower dosages can also contribute to reducing the likelihood of side-effects caused by higher dosages. Additionally, the composition allows for the administration of bupropion and/or phentermine over a prolonged period of time with less risk of dependence. Over time and/or if tolerance develops, the dosage of bupropion and/or the dosage of phentermine can be increased for the continued treatment of pain such as, for example, chronic pain, neuropathic pain and nociceptive pain. Thus, the lower initial dosages of bupropion and phentermine in the composition of the present disclosure can allow for a longer treatment period than when bupropion and phentermine are administered in their single agent dosages.

In one embodiment, the composition of the present disclosure includes a therapeutically effective amount of bupropion and a therapeutically effective amount of phentermine As used herein, “therapeutically effective amount” refers to that amount that provides a therapeutic effect for a given condition and administration regimen. It can be understood, however, that the total daily usage of the compounds and compositions of the disclosure can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. Actual dosage levels of active ingredients in the compositions disclosed herein can be varied so as to obtain an amount of the active compound(s) that is effective to achieve the desired response for a particular individual, composition and mode of administration. The selected dosage level can depend upon the activity of the particular compound, the route of administration, the severity of the condition being treated and the condition and prior medical history of the individual being treated. However, it is within the skill of the art to start doses of the compound at levels lower than required to achieve the desired effect and to gradually increase the dosage until the desired effect is achieved. Upon achieving the desired effect, a fixed ratio of each agent can be determined to accommodate a pill, capsule or tablet such that the composition can be administered over a period of time such as, for example, a 24 hour period.

The active compounds (e.g., bupropion and phentermine) can be administered in a convenient manner such as by oral administration, injection (subcutaneous, intravenous, etc.), inhalation, transdermal application, or rectal administration. A preferred route of administration is by oral administration. Depending on the route of administration, the active compound may be coated in a material to protect the compound from the action of enzymes, acids and other natural conditions which may inactivate the compound.

As used herein, “dosage” refers to physically discrete units suited as unitary dosages for the individual to be treated; each unit containing a predetermined quantity of active compound calculated to produce the desired effect in association with the required pharmaceutical carrier. The specification for the dosage unit forms of the present disclosure are dictated by and directly dependent on (a) the unique characteristics of the active compound and the particular effect to be achieved, and (b) the limitations inherent in the art of compounding such an active compound for the treatment of an individuals.

In administering the composition of the present disclosure, the same dose that is usually given as a single agent may be given through a normal administration route. In a particularly preferred embodiment, a reduced dose (for example, 0.10-0.99 times the highest dose as a single agent) can be given through a normal administration route.

A particularly suitable composition for oral administration can be a pill-form (for example, a tablet and a capsule) containing bupropion and phentermine The dosage of bupropion can be, for example, from about 75 mg to about 400 mg, including from about 75 mg to about 100 mg, from about 75 mg to about 150 mg, from about 75 mg to about 200 mg, and from about 75 mg to about 300 mg. The dosage of phentermine can be, for example, from about 8 mg to about 37.5 mg, including from about 8 mg to about 10 mg, from about 8 mg to about 15 mg, and from about 8 mg to about 30 mg. In one particularly suitable embodiment, the composition can include about 400 mg of bupropion and from about 8 mg phentermine to about 37.5 mg phentermine. In another particularly suitable embodiment, the composition can include about 400 mg of bupropion and about 8 mg phentermine In another particularly suitable embodiment, the composition can include about 400 mg of bupropion and about 10 mg phentermine. In another particularly suitable embodiment, the composition can include about 400 mg of bupropion and about 15 mg phentermine. In another particularly suitable embodiment, the composition can include about 400 mg of bupropion and about 30 mg phentermine In another particularly suitable embodiment, the composition can include about 400 mg of bupropion and about 37.5 mg phentermine. For adults, the dosages of methylphenidate (RITALIN®) can be from about 10 mg/day to about 60 mg/day and can be administered in two to three divided doses per day. For adolescents age 13 to 17 years, the dosages of methylphenidate (CONCERTA®) can be from about 18 mg/day to about 72 mg/day. For adults 18 years and older, the dosages of methylphenidate (CONCERTA®) can be from about 18 mg/day to about 72 mg/day. For adults, the dosage of atomoxetine can be from about 20 mg/day to about 100 mg/day and can be administered in 2 divided doses per day.

For children age 6 years and older, the dosage of bupropion can be from about 1 mg/kg of body weight per day to about 6 mg/kg of body weight per day. For children age 6 years and older with a body weight up to 70 kg, the dosages of atomoxetine can be from about 0.5 mg/kg of body weight per day to about 1.4 mg/kg of body weight per day. For children age 6 years and older with a body weight over 70 kg, the dosages of atomoxetine can be from about 20 mg/day to about 40 mg/day. For children age 6 years and older, the dosages of methylphenidate (RITALIN®) can be from about 0.3 mg/kg of body weight per day to about 2 mg/kg of body weight per day in three (3) divided doses or from about 5 mg twice per day to about 30 mg twice per day up to 60 mg per day. For children age 6 to 12 years, the dosages of methylphenidate (CONCERTA®) can be from about 18 mg/day to about 54 mg/day.

The individual in need can be diagnosed with chronic pain and/or suspected of having chronic pain. The individual in need can be diagnosed with neuropathic pain and/or suspected of having neuropathic pain. The individual in need can be diagnosed with nociceptive pain and/or suspected of having nociceptive pain. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein.

As used herein, the term “individual” refers to a male human or a female human. The individual can be, for example, a human. The individual can be an adult individual, an adolescent child (age 6 to 18 years), a child (less than 6 years), and a pediatric individual.

The effect of administering the composition including bupropion and phentermine can be confirmed by magnetic resonance imaging of the brain areas related to the processing of pain as can be determined and/or observed by one skilled in the art such as, for example, medical professionals. Electroencephalography can also be used to analyze brain activity to determine the effect of administering the combination therapy described herein.

The composition can be administered in any manner effective for delivering the compounds forming the combination. Exemplary modes of administration may be, for example, orally, topically, transdermally, parenterally, enterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. Particularly suitable administration is orally.

The compounds described herein are administered as a combination. It will be appreciated that when using the composition, the compounds may be in the same pharmaceutically acceptable carrier at a specific ratio that is administered simultaneously in the same pharmaceutically acceptable carrier to achieve the desired effect. Buproprion and its metabolites such as, for example, R,R-hydroxybupropion, S,S-hydroxybupropion, threo-hydrobupropion and erythro-hydrobupropion, can be monitored for example, by measurement in the blood and using positron emission tomography of the brain. Similarly, phentermine and its metabolites can be measured in the blood. The ratio of each compound can be determined and/or adjusted by one skilled in the art using methods known by those skilled in the art such as, for example, measuring buproprion and phentermine and their metabolites in the blood. Thus, the individual receives the composition containing the specific ratio of each compound to achieve the desired effect. The composition can also be in separate pharmaceutical carriers such as conventional dosage forms that are administered simultaneously such that each compound is administered at the specific ratio. For example, the compounds forming the composition can be in separate pill forms that are co-administered to the individual. Administration of each compound may be effected by any appropriate route such as, for example, oral routes, intravenous routes, intramuscular routes, and direct absorption through mucous membrane tissues. The compounds may be administered by the same route or by different routes. For example, a first compound of the composition may be administered orally while the other compound of the composition may be administered by a non-oral route. The amounts of each compound are administered at a dosage of each to arrive at the specific ratio desired to achieve the desired effect. Additionally, for example, one or more compounds can be used in an intravenous drip in combination with one or more compounds administered as a transdermal patch or paste.

The composition can also include a pharmaceutically acceptable carrier. The term “pharmaceutically acceptable carrier” refers to any suitable adjuvant, carrier, excipient, additive, antioxidant, osmolality adjusting agent, buffer, pH adjusting agent, or stabilizer. The pharmaceutically acceptable carrier may be in solid or liquid form such as, tablets, capsules, powders, solutions, suspensions, or emulsions. Particularly suitable pharmaceutically carriers may be, for example, saline (solution of 0.90% w/v of NaCl), half-normal saline (solution of 0.45% NaCl), quarter-normal saline (solution of 0.22% NaCl), D5W (a 5% dextrose in water solution), DSNS (a 5% dextrose in normal saline solution), Ringer's lactate, and other solutions known by those skilled in the art.

In view of the above, it will be seen that the several advantages of the disclosure are achieved and other advantageous results attained. As various changes could be made in the above methods without departing from the scope of the disclosure, it is intended that all matter contained in the above description shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

### EXAMPLES

### Example 1

In this Example, a composition including bupropion and phentermine was administered to an individual for treating pain.

In particular the individual suffered from low back pain and plantar faciitis. A combination of 200 mg of bupropion and 18.75 mg of phentermine was administered to a male individual over a 3 week period. Results showed a good pain relief and the individual did not require other pain medication.

1. A method for treating chronic pain in an individual in need comprising:
administering to the individual a composition that comprises methylphenidate, bupropion and phentermine, wherein the bupropion comprises a dosage of from about 75 mg to about 400 mg and the phentermine comprises a dosage of from about 8 mg to about 37.5 mg.

2-3. (canceled)

4. The method of claim 1, wherein the composition further comprises a pharmaceutically acceptable carrier.

5. The method of claim 1, wherein the administering is selected from the group consisting of orally, topically, transdermally, parenterally, subcutaneously, intravenously, intramuscularly, intraperitoneally, by intranasal instillation, by intracavitary or intravesical instillation, intraocularly, intraarterially, intralesionally, application to mucous membranes and combinations thereof.

6. A method for treating neuropathic pain in an individual in need comprising:
administering to the individual a combination therapy that comprises methylphenidate, bupropion and phentermine, wherein the bupropion comprises a dosage of from about 75 mg to about 400 mg and the phentermine comprises a dosage of from about 8 mg to about 37.5 mg.

7-8. (canceled)

9. The method of claim 6, wherein the composition further comprises a pharmaceutically acceptable carrier.

10. The method of claim 6, wherein the administering is selected from the group consisting of orally, topically, transdermally, parenterally, enterally, subcutaneously, intravenously, intramuscularly, intraperitoneally, by intranasal instillation, by intracavitary or intravesical instillation, intraocularly, intraarterially, intralesionally, application to mucous membranes and combinations thereof.

11. A method for treating nociceptive pain in an individual in need comprising:
administering to the individual a combination therapy that comprises bupropion and methylphenidate, wherein the bupropion comprises a dosage of from about 75 mg to about 400 mg and the methylphenidate comprises a dosage of from about 10 mg/day to about 72 mg/day.

12-13. (canceled)

14. The method of claim 11, wherein the composition further comprises a pharmaceutically acceptable carrier.

15. The method of claim 11, wherein the administering is selected from the group consisting of orally, topically, transdermally, parenterally, enterally, subcutaneously, intravenously, intramuscularly, intraperitoneally, by intranasal instillation, by intracavitary or intravesical instillation, intraocularly, intraarterially, intralesionally, application to mucous membranes and combinations thereof.

16. The method of claim 1, wherein the bupropion comprises a dosage of from about 75 mg to about 100 mg.

17. The method of claim 1, wherein the phentermine comprises a dosage of from about 8 mg to about 30 mg.

18. The method of claim 1, wherein the bupropion comprises a dosage of from about 75 mg to about 200 mg and the phentermine comprises a dosage of from about 8 mg to about 30 mg.

19. (canceled)

20. The method of claim 6, wherein the bupropion comprises a dosage of from about 75 mg to about 100 mg.

21. The method of claim 6, wherein the phentermine comprises a dosage of from about 8 mg to about 30 mg.

22. The method of claim 6, wherein the bupropion comprises a dosage of from about 75 mg to about 200 mg and the phentermine comprises a dosage of from about 8 mg to about 30 mg.

23. The method of claim 6, wherein the bupropion comprises a dosage of about 400 mg.

24. The method of claim 11, wherein the bupropion comprises a dosage of from about 75 mg to about 100 mg.

25. The method of claim 11, wherein the therapy further comprises administering phentermine, wherein the phentermine comprises a dosage of from about 8 mg to about 30 mg.

26. (canceled)

27. The method of claim 1, wherein the methylphenidate comprises a dosage of from about 10 mg/day to about 72 mg/day.

28. The method of claim 6, wherein the methylphenidate comprises a dosage of from about 10 mg/day to about 72 mg/day.
